(19) United States
(12) Patent Application Publication
(10) Pub. No.: US 2011/0190345 A1
(43) Pub. Date: Aug. 4, 2011

(54) Treatment of Psychosis with a 5HT2A Antagonist and a Metabotropic Glutamate Receptor Agonist or Potentiator

(76) Inventors: Sarah L. Huszar, Lansdale, NJ (US); Pete Haynes Hutson, Lansdale, PA (US); Jason M. Uslaner, Berwyn, PA (US)

(21) Appl. No.: 12/668,063

(22) PCT Filed: Aug. 1, 2008
(86) PCT No.: PCT/US08/09340

§ 371 Date: Sep. 16, 2010

Related U.S. Application Data
(60) Provisional application No. 60/963,625, filed on Aug. 6, 2007.

(51) Int. Cl. A61K 31/445; A61P 25/18

(52) U.S. Cl. 514/317

(57) ABSTRACT

The present invention is directed to the use of a 5-HT2A antagonist and an mGluR2/3 agonist, an mGluR2 agonist or an mGluR2 potentiator for the treatment of psychosis, including schizophrenia or bipolar disorder.

### BACKGROUND OF THE INVENTION

Schizophrenia is among the most debilitating psychiatric disorders and has a considerable social and economic impact as it affects approximately 1% of the world's population. Indeed, schizophrenia is more prevalent than Alzheimer's disease, multiple sclerosis and even diabetes. In the United States, around 2.5 million persons are affected with a cost of $40 billion/year if productivity losses are included. The essential features of schizophrenia are a mixture of characteristic signs and symptoms (both positive and negative) which are present in an individual for a significant portion of time over at least one month. The so-called “active-phase” symptoms include delusions, hallucinations, disorganized speech, disorganized or catatonic behavior and negative symptoms (e.g. affective flattening, alogia and avolition). Some patients have only a single episode of the illness, but most have either recurrent episodes or chronic illness. While the etiology of schizophrenia is currently unknown, the disease appears to be produced by a complex interaction of biological, environmental, and genetic factors.

Numerous compounds have been developed for treating disorders characterized by psychotic symptoms, including e.g., sedatives, hypnotics, typical antipsychotics, atypical antipsychotics, and the like. However, up to 30% of schizophrenic patients are not adequately treated by currently available medication and these medications suffer from a high rate of discontinuation due to either patient and/or physician dissatisfaction with efficacy or safety/tolerability. Currently used typical and atypical antipsychotic treatments have a number of major drawbacks that include significant side effect liability as well as lack of complete efficacy in ameliorating psychotic symptoms. The typical antipsychotics possess antipsychotic and sedative properties to varying degrees and are generally effective against positive symptoms, but are not generally effective against negative symptoms and may even exacerbate them. Typical antipsychotic drugs also have a propensity to induce disabling and ultimately disfiguring Parkinson-like extrapyramidal motor symptoms, such as tardive dyskinesia. Treatment of schizophrenia with antipsychotic (or neuroleptic) agents, such as haloperidol and chlorpromazine, is associated with a number of side-effects, including extrapyramidal symptoms, acute dystonias, tardive dyskinesias, akathesia, tremor, tachycardia, drowsiness, confusion, postural hypotension, blurring of vision, precipitation of glaucoma, dry mouth, constipation, urinary hesitance and impaired sexual function. Such side-effects are often debilitating and contribute considerably to a patient's non-compliance with prescribed treatment. They may also hinder a patient's social rehabilitation. The atypical antipsychotics offer modest efficacy against negative symptoms and relatively improved tolerability with respect to extrapyramidal motor symptoms, however, atypical antipsychotics are also associated by a variety of side-effects, including severe metabolic disorders and weight gain. In view of the short-comings of existing antipsychotic therapy, and the inability of the currently prevailing dopamine hyperfunction hypothesis to fully account for the pathophysiology of schizophrenia, there is a need for antipsychotic agents with better efficacy on positive symptoms, negative symptoms and/or decreased adverse side effects.

Receptors for serotonin (5-hydroxytryptamine, 5-HT) are an important class of G protein-coupled receptors. Serotonin is thought to play a role in processes related to learning and memory, sleep, thermoregulation, mood, motor activity, pain, sexual and aggressive behaviours, appetite, neurodegenerative regulation, and biological rhythms. Serotonin receptors are divided into seven subfamilies, referred to as 5-HT1 through 5-HT7, inclusive. These subfamilies are further divided into subtypes. For example, the 5-HT2 subfamily is divided into three receptor subtypes: 5-HT2A, 5-HT2B and 5-HT2C. The human 5-HT2C receptor was first isolated and cloned in 1987, and the human 5-HT2A receptor was first isolated and cloned in 1990.

The excitatory amino acid L-glutamate (sometimes referred to herein simply as glutamate) through its many receptors mediates most of the excitatory neurotransmission within the mammalian central nervous system (CNS). The excitatory amino acids, including glutamate, are of great physiological importance, playing a role in a variety of physiological processes, such as long-term potentiation (learning and memory), the development of synaptic plasticity, motor control, respiration, cardiovascular regulation, and sensory perception. Glutamate acts via at least two distinct classes of receptors. One class is composed of the ionotropic glutamate (iGlu) receptors that act as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. The mGluR receptors belong to the Type III G-protein coupled receptor (GPCR) superfamily. This superfamily of GPCR's include the calcium-sensing receptors, GABAB receptors and pheromone receptors, which are unique in that they are activated by binding of effectors to the amino-terminus portion of the receptor protein. The mGlu receptors are thought to mediate glutamate's demonstrated ability to modulate intracellular signal transduction pathways. They have been demonstrated to be localized both pre- and post-synaptically where they can regulate neurotransmitter release, either glutamate or other neurotransmitters, or modify the post-synaptic response of neurotransmitters, respectively.

There are eight distinct mGlu receptors that have been positively identified, cloned, and their sequences reported. These are further subdivided based on their amino acid sequence homology, their ability to effect certain signal transduction mechanisms, and their known pharmacological properties. For instance, the Group I mGluR receptors, which include the mGlu1R and mGlu5R, are known to activate phospholipase C (PLC) via Gaq-proteins thereby resulting in the increased hydrolysis of phosphoinositides and intracellular calcium mobilization. There are several compounds that are reported to activate the Group I mGlu receptors including DHPG, (R/S)-3,5-dihydroxyphenylglycine. The Group II mGlu receptors (Gi/cAMP Coupled) consist of the two distinct receptors, mGluR2 and mGluR3 receptors. Both have been found to be negatively coupled to adenylate cyclase via activation of Gi-protein. These receptors can be activated by a selective compound such as 1S,2S,SR,6S-2 aminobicyclo[3.1.0]hexane-2,6-dicarboxylate (Monn, et al., J. Med. Chem., 40, 528 (1997); Schoepp, et al., Neuropharmacol., 36, 1 (1997)). Similarly, the Group III mGlu receptors, including mGluR4, mGluR6, mGluR7 and mGluR8, are negatively coupled to adenylate cyclase via Gai and are potently activated by L-AP4 (L-(+)-2-amino-4-phosphonobutyric acid). (Schoepp, Neurochem. Int., 24, 439 (1994)).

The present invention helps to meet the need for providing antipsychotic agents with better efficacy on positive symptoms, negative symptoms and/or decreased adverse side effects. In particular, the use of a serotonin 5HT2A antagonist and an mGluR2 agonist, an mGluR2/3 agonist or an mGluR2 potentiator surprisingly provides greater efficacy and/or decreased adverse side effects in the treatment of psychosis than each agent alone.

### SUMMARY OF THE INVENTION

The present invention is directed to the use of a 5HT2A antagonist and an mGluR2/3 agonist, an mGluR2 agonist or an mGluR2 potentiator for the treatment of psychosis, such as a schizophrenic disorder or psychosis in Alzheimer's disease or bipolar disorder.

### BRIEF DESCRIPTION OF THE DRAWINGS

FIG. 1. A bar chart showing locomotor response of male Wistar rats over a 90 minute session after receiving ketamine or saline as a function of treatment with vehicle, a 5-HT2a antagonist (0.2 mpk), an mGluR2/3 agonist (1 mpk), or the 5-HT2a antagonist (0.2 mpk)+the mGluR2/3 agonist (1 mpk).

FIG. 2. A bar chart showing locomotor response of male Wistar rats over the first 60 minutes after receiving ketamine or saline as a function of treatment with vehicle, a 5-HT2a antagonist (0.2 mpk), an mGluR2/3 agonist (1 mpk), or the 5-HT2a antagonist (0.2 mpk)+the mGluR2/3 agonist (1 mpk).

FIG. 3. A bar chart showing locomotor response of male Wistar rats over a 90 minute session after receiving amphetamine (1.5 mg/kg) or saline as a function of treatment with vehicle, a 5-HT2a antagonist (0.2 mpk), an mGluR2/3 agonist (1 mpk), or the 5-HT2a antagonist (0.2 mpk)+the mGluR2/3 agonist (1 mpk).

### DETAILED DESCRIPTION OF THE INVENTION

The present invention is directed to the use of a 5HT2A antagonist and an mGluR2/3 agonist, an mGluR2 agonist or an mGluR2 potentiator for the treatment of psychosis, such as a schizophrenic disorder or psychosis in Alzheimer's disease or bipolar disorder. The present invention further provides a combination which comprises a 5-HT2A antagonist or a pharmaceutically acceptable salt thereof, and an mGluR2/3 agonist, an mGluR2 agonist or an mGluR2 potentiator or a pharmaceutically acceptable salt thereof. In an embodiment, the combination of the present invention comprises an mGluR2/3 agonist or a pharmaceutically acceptable salt thereof. In an embodiment, the combination of the present invention comprises an mGluR2 agonist or a pharmaceutically acceptable salt thereof. In an embodiment, the combination of the present invention comprises an mGluR2 potentiator or a pharmaceutically acceptable salt thereof.

In another embodiment, the present invention provides a pharmaceutical composition which comprises a pharmaceutically acceptable carrier, a 5-HT2A antagonist or a pharmaceutically acceptable salt thereof, and an mGluR2/3 agonist, an mGluR2 agonist or an mGluR2 potentiator or a pharmaceutically acceptable salt thereof. The present invention also provides the use of a 5-HT2A antagonist or a pharmaceutically acceptable salt thereof, and an mGluR2/3 agonist, an mGluR2 agonist or an mGluR2 potentiator or a pharmaceutically acceptable salt thereof, for the manufacture of a medicament for treating a psychotic disorder, such as a schizophrenic disorder or psychosis in Alzheimer's disease or bipolar disorder. The present invention also provides a kit comprising a pharmaceutical composition which comprises a pharmaceutically acceptable carrier, a 5-HT2A antagonist or a pharmaceutically acceptable salt thereof, and an mGluR2/3 agonist, an mGluR2 agonist or an mGluR2 potentiator or a pharmaceutically acceptable salt thereof together with instructions for administering said medicaments sequentially or simultaneously to a patient suffering from a psychotic disorder, such as a schizophrenic disorder or psychosis in Alzheimer's disease or bipolar disorder. The present invention also provides a product comprising a therepeutically effective amount of a 5-HT2A antagonist or a pharmaceutically acceptable salt thereof, and a therepeutically effective amount of an mGluR2/3 agonist, an mGluR2 agonist or an mGluR2 potentiator or a pharmaceutically acceptable salt thereof, as a combined preparation for simultaneous, separate or sequential use in the treatment of a psychotic disorder, such as a schizophrenic disorder or psychosis in Alzheimer's disease or bipolar disorder. The present invention also provides products for containing a therepeutically effective amount of a 5-HT2A antagonist or a pharmaceutically acceptable salt thereof, and a therepeutically effective amount of an mGluR2/3 agonist, an mGluR2 agonist or an mGluR2 potentiator or a pharmaceutically acceptable salt thereof, as a combined preparation for simultaneous, separate or sequential use in the treatment of a psychotic disorder, such as a schizophrenic disorder or psychosis in Alzheimer's disease or bipolar disorder.

The present invention is also directed to a method for treating psychosis in a mammalian patient in need thereof which comprises administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating schizophrenia in a mammalian patient in need thereof which comprises administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating bipolar disorder in a mammalian patient in need thereof which comprises administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating Alzheimer's disease, including psychosis in Alzheimer's disease, in a mammalian patient in need thereof which comprises administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for enhancing cognition in a mammalian patient in need thereof which comprises administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating psychosis in a mammalian patient in need thereof which comprises the step of administering to the patient a therapeutically effective amount of a combination of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating schizophrenia in a mammalian patient in need thereof which comprises the step of administering to the patient a therapeutically effective amount of a combination of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating bipolar disorder in a mammalian patient in need thereof which comprises the step of administering to the patient a therapeutically effective amount of a combination of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating Alzheimer's disease, including psychosis in Alzheimer's disease, in a mammalian patient in need thereof which comprises the step of administering to the patient a therapeutically effective amount of a combination of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for enhancing cognition in a mammalian patient in need thereof which comprises the step of administering to the patient a therapeutically effective amount of a combination of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating psychosis which comprises: diagnosing a mammalian patient as being in need of thereof; and administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating schizophrenia which comprises: diagnosing a mammalian patient as being in need of thereof; and administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating bipolar disorder which comprises: diagnosing a mammalian patient as being in need of thereof; and administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for treating Alzheimer's disease, including psychosis in Alzheimer's disease, which comprises: diagnosing a mammalian patient as being in need of thereof; and administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

The present invention is also directed to a method for enhancing cognition which comprises: diagnosing a mammalian patient as being in need of thereof; and administering to the patient a therapeutically effective amount of a 5-HT2A antagonist or a pharmaceutically acceptable salt thereof, and an mGluR2/3 agonist, an mGluR2 agonist or an mGluR2 potentiator or a pharmaceutically acceptable salt thereof.

By the term “5-HT2A antagonist” is meant any exogenously administered compound or agent that directly or indirectly attenuates the activity of agonists at the 5-HT2A receptor, in particular, the human 5-HT2A receptor. 5-HT2A antagonists suitable for use in the present invention may be identified without undue experimentation by methodology well known in the art (see for example Fletcher et al, J. Med. Chem., 2002, 45, 492-503). In an aspect of the present invention, the 5-HT2A antagonist is selective for the human 5-HT2A receptor over other receptors, notably the human 5-HT2C and other serotonin receptors, dopamine receptors, and IKr (voltage-dependent potassium channel). In an aspect of the present invention, the 5-HT2A antagonist is selective for the human 5-HT2A receptor over the dopamine D2 receptor.

In an aspect of the present invention, the 5-HT2A antagonist is selective for the human 5-HT2A receptor with a pKi value equal to or higher than 8 towards the 5-HT2A receptor and less than 8 towards other 5HT receptors. In an aspect of the present invention, the 5-HT2A antagonist possesses a selectivity for the 5-HT2A receptor relative to each of the other 5-HT2 receptors of at least 5 fold as measured by the ratio of IC50 for the 5-HT2A receptor to the 1050 for each of the other 5-HT2 receptors. In an aspect of the present invention, the 5-HT2A antagonist possesses a selectivity for the 5-HT2A receptor relative to each of the other 5-HT2 receptors of at least 10 fold as measured by the ratio of IC50 for the 5-HT2A receptor to the IC50 for each of the other 5-HT2 receptors. In an aspect of the present invention, the 5-HT2A antagonist possesses a selectivity for the 5-HT2A receptor relative to each of the other 5-HT2 receptors of at least 50 fold as measured by the ratio of IC50 for the 5-HT2A receptor to the IC50 for each of the other 5-HT2 receptors. In an aspect of the present invention, the 5-HT2A antagonist possesses a selectivity for the 5-HT2A receptor relative to each of the other 5-HT2 receptors of at least 100 fold as measured by the ratio of IC50 for the 5-HT2A receptor to the IC50 for each of the other 5-HT2 receptors.

In an aspect of the present invention, the 5-HT2A antagonist possesses a selectivity for the 5-HT2A receptor relative to the dopamine D2 receptor of at least 5 fold as measured by the ratio of IC50 for the 5-HT2A receptor to the IC50 for the dopamine D2 receptor. In an aspect of the present invention, the 5-HT2A antagonist possesses a selectivity for the 5-HT2A receptor relative to the dopamine D2 receptor of at least 10 fold as measured by the ratio of IC50 for the 5-HT2A receptor to the IC50 for the dopamine D2 receptor. In an aspect of the present invention, the 5-HT2A antagonist possesses a selectivity for the 5-HT2A receptor relative to the dopamine D2 receptor of at least 50 fold as measured by the ratio of IC50 for the 5-HT2A receptor to the IC50 for the dopamine D2 receptor. In an aspect of the present invention, the 5-HT2A antagonist possesses a selectivity for the 5-HT2A receptor relative to the dopamine D2 receptor of at least 100 fold as measured by the ratio of IC50 for the 5-HT2A receptor to the IC50 for the dopamine D2 receptor.

In an aspect of the present invention, the 5-HT2A antagonist possesses a selectivity for the 5-HT2A receptor relative to the IKr (voltage-dependent potassium channel) of at least 5 fold as measured by the ratio of IC50 for the 5-HT2A receptor to the IC50 for the IKr (voltage-dependent potassium channel). In an aspect of the present invention, the 5-HT2A antagonist possesses a selectivity for the 5-HT2A receptor relative to the IKr (voltage-dependent potassium channel) of at least 10 fold as measured by the ratio of IC50 for the 5-HT2A receptor to the IC50 for the IKr (voltage-dependent potassium channel). In an aspect of the present invention, the 5-HT2A antagonist possesses a selectivity for the 5-HT2A receptor relative to the IKr (voltage-dependent potassium channel) of at least 50 fold as measured by the ratio of IC50 for the 5-HT2A receptor to the IC50 for the IKr (voltage-dependent potassium channel). In an aspect of the present invention, the 5-HT2A antagonist possesses a selectivity for the 5-HT2A receptor relative to the IKr (voltage-dependent potassium channel) of at least 100 fold as measured by the ratio of IC50 for the 5-HT2A receptor to the IC50 for the IKr (voltage-dependent potassium channel).

In an aspect of the present invention the 5-HT2A antagonist possesses an IC50 for blocking the 5-HT2A antagonist receptor of 500 nM or less as evaluated by the above assay. In an aspect of the present invention the 5-HT2A antagonist possesses an IC50 for blocking the 5-HT2A antagonist receptor of 100 nM or less as evaluated by the above assay. In an aspect of the present invention the 5-HT2A antagonist possesses an IC50 for blocking the 5-HT2A antagonist receptor of 50 nM or less as evaluated by the above assay. In an aspect of the present invention the 5-HT2A antagonist possesses an IC50 for blocking the 5-HT2A antagonist receptor of 10 nM or less as evaluated by the above assay.

In an embodiment of the present invention the 5-HT2A antagonist is an orally active 5-HT2A antagonist. In an embodiment of the present invention the 5-HT2A antagonist is orally administered. In another embodiment of the present invention the 5-HT2A antagonist is a non-peptidal 5-HT2A antagonist. In an embodiment of the present invention the 5-HT2A antagonist is a CNS-penetrant 5-HT2A antagonist and is able to enter the brain and/or central nervous system with sufficient concentration to have a therapeutic effect. In a further embodiment of the present invention the CNS-penetrant 5-HT2A antagonist is a compound that exhibits sufficient concentration in the brain and/or central nervous system to have therapeutic efficacy upon oral administration.

In an embodiment of the present invention the 5-HT2A antagonist has an onset of action of 45-60 minutes. In an embodiment of the present invention the 5-HT2A antagonist has a pharmacological half life (T½ life) of short duration. In another embodiment of the present invention the 5-HT2A antagonist has a pharmacological half life (T½ life) of intermediate duration. In another embodiment of the present invention the 5-HT2A antagonist has a pharmacological half life (T½ life) of long duration. In another embodiment of the present invention the 5-HT2A antagonist has a pharmacological half life (T½ life) of at least about 2 hours duration.

5-HT2A antagonists of use in the present invention are disclosed in e.g., WO 91/18602, WO 98/38189, WO 99/00119, WO 99/11619, WO 99/11641, WO 99/47511, WO 00/04017, WO 00/05229, WO 00/107435, WO 00/166521, WO 01/51469, WO 00/43362, WO 00/77010, WO 01/74794, WO 2004/058722, WO 2004/101518, WO 2005/047246, WO 2005/047247, WO 2006/013397, WO 2006/021806, WO 2006/059149, WO 2006/097766, WO 2006/095205, WO 2006/100519, EP 0208235, EP 0373998, US 2003/0166928, US 2004/0106600, U.S. Pat. No. 5,134,149, U.S. Pat. No. 5,169,096, U.S. Pat. No. 6,028,083, U.S. Pat. No. 6,063,793, U.S. Pat. No. 6,486,153, U.S. Pat. Nos. 6,559,166, 6,756,393, U.S. Pat. No. 6,777,430, U.S. Pat. No. 7,094,777, U.S. Pat. No. 7,217,740. Specific examples of 5-HT2A antagonists include AVE-8488 (Sanofi-aventis), BVT-28949 (Biovitrum), eplivanserin (WO 99/00119), lubazodone, Org-50081, pimavanserin (WO 00/166521), pruvanserin (WO 00/107435), SR 46349B and volinanserin (MDL 100,907), or a pharmaceutically acceptable salt thereof. Eplivanserin can be prepared as described in WO 99/00119. Lubazodone has the chemical name (S)-2-[[(7-fluoro-2,3 dihydro-1H-inden-4-yl)-oxy]methyl]morpholine hydrochloride and can be prepared as described in Eur. J. Pharmacol. 1997, 329, 27-35. Pimavanserin can be prepared as described in WO 00/166521. Pruvanserin can be prepared as described in WO 00/107435. SR 46349B has the chemical name (1Z,2E)-1-(2-fluorophenyl)-3-(4-hydroxyphenyl)prop-2-en-1-one-O-(2-dimethyl-aminoethyl)oxime hemifumarate and can be prepared as described in EP 0373998. Volinanserin (MDL 100,907) has the chemical name (+)-alpha-(2,3-dimethoxyphenyl)-1-[2-(4 fluorophenyl)ethyl]-4-piperidine methanol and can be prepared as described in U.S. Pat. No. 5,134,149 and WO 91/18602. Compounds that are structurally similar to MDL 100,907 are described in EP 0208235, and ester derivatives of MDL 100,907 that function as prodrugs for MDL 100,907 are described in U.S. Pat. Nos. 6,028,083 and 6,063,793.

By the term “mGluR2/3 agonist” is meant any exogenously administered compound or agent that augments the activity of the Group II (Gi/cAMP Coupled) mGlu receptors (in particular, the mGluR2 receptor and the mGluR3 receptor), such as the human mGluR2 receptor and the human mGluR3 receptor. mGluR2/3 agonists suitable for use in the present invention may be identified without undue experimentation by methodology well known in the art (see for example Monn et al., (1999) J. Med Chem 42(6), 1027-1040.)

In an embodiment of the present invention the mGluR2/3 agonist possesses a selectivity for the mGluR2 receptor and the mGluR3 receptor relative to ionotropic glutamate receptors of at least 5 fold as measured by the ratio of EC50 for the mGluR2 receptor and the mGluR3 receptor to the EC50 for ionotropic glutamate receptors as evaluated by the above assays. In an embodiment of the present invention the mGluR2/3 agonist possesses a selectivity for the mGluR2 receptor and the mGluR3 receptor relative to ionotropic glutamate receptors of at least 10 fold as measured by the ratio of EC50 for the mGluR2 receptor and the mGluR3 receptor to the EC50 for ionotropic glutamate receptors as evaluated by the above assays. In an embodiment of the present invention the mGluR2/3 agonist possesses a selectivity for the mGluR2 receptor and the mGluR3 receptor relative to ionotropic glutamate receptors of at least 50 fold as measured by the ratio of EC50 for the mGluR2 receptor and the mGluR3 receptor to the EC50 for ionotropic glutamate receptors as evaluated by the above assays. In an embodiment of the present invention the mGluR2/3 agonist possesses a selectivity for the mGluR2 receptor and the mGluR3 receptor relative to ionotropic glutamate receptors of at least 100 fold as measured by the ratio of EC50 for the mGluR2 receptor and the mGluR3 receptor to the EC50 for ionotropic glutamate receptors as evaluated by the above assayss.

In an embodiment of the present invention the mGluR2/3 agonist possesses a selectivity for the mGluR2 receptor and the mGluR3 receptor relative to each of the other mGluR receptors of at least 5 fold as measured by the ratio of EC50 for the mGluR2 receptor and the mGluR3 receptor to the EC50 for each of the other mGluR receptors as evaluated by the above assays. In an embodiment of the present invention the mGluR2 agonist possesses a selectivity for the mGluR2 receptor and the mGluR3 receptor relative to each of the other mGluR receptors of at least 10 fold as measured by the ratio of EC50 for the mGluR2 receptor and mGluR3 receptor to the EC50 for each of the other mGluR receptors as evaluated by the above assays. In an embodiment of the present invention the mGluR2 agonist possesses a selectivity for the mGluR2 receptor and the mGluR3 receptor relative to each of the other mGluR receptors of at least 50 fold as measured by the ratio of EC50 for the mGluR2 receptor and the mGluR3 receptor to the EC50 for each of the other mGluR receptors as evaluated by the above assays. In an embodiment of the present invention the mGluR2 agonist possesses a selectivity for the mGluR2 receptor and the mGluR3 receptor relative to each of the other mGluR receptors of at least 100 fold as measured by the ratio of EC50 for the mGluR2 receptor and the mGluR3 receptor to the EC50 for each of the other mGluR receptors as evaluated by the above assays.

In an aspect of the present invention the mGluR2/3 agonist possesses an EC50 for binding to the mGluR2 receptor and the mGluR3 receptor of 1 uM or less as evaluated by the above assays. In an aspect of the present invention the mGluR2/3 agonist possesses an EC50 for binding to the mGluR2 receptor and the mGluR3 receptor of 500 nM or less as evaluated by the above assays. In an aspect of the present invention the mGluR2/3 agonist possesses an EC50 for binding to the mGluR2 receptor and the mGluR3 receptor of 100 nM or less as evaluated by the above assays. In an aspect of the present invention the mGluR2/3 agonist possesses an EC50 for binding to the mGluR2 receptor and the mGluR3 receptor of 50 nM or less as evaluated by the above assays. In an aspect of the present invention the mGluR2/3 agonist possesses an EC50 for binding to the mGluR2 receptor and the mGluR3 receptor of 10 nM or less as evaluated by the above assays.

In an embodiment of the present invention the mGluR2/3 agonist is an orally active mGluR2/3 agonist. In an embodiment of the present invention the mGluR2/3 agonist is orally administered. In another embodiment of the present invention the mGluR2/3 agonist is a non-peptidal mGluR2/3 agonist. In an embodiment of the present invention the mGluR2/3 agonist is a CNS-penetrant mGluR2/3 agonist and is able to enter the brain and/or central nervous system with sufficient concentration to have a therapeutic effect. In a further embodiment of the present invention the CNS-penetrant mGluR2/3 agonist is a compound that exhibits sufficient concentration in the brain and/or central nervous system to have therapeutic efficacy upon oral administration.

In an embodiment of the present invention the mGluR2/3 agonist has an onset of action of 45-60 minutes. In an embodiment of the present invention the mGluR2/3 agonist has a pharmacological half life (T½ life) of short duration. In another embodiment of the present invention the mGluR2/3 agonist has a pharmacological half life (T½ life) of intermediate duration. In another embodiment of the present invention the mGluR2 agonist has a pharmacological half life (T½ life) of long duration. In another embodiment of the present invention the mGluR2/3 agonist has a pharmacological half life (T½ life) of at least about 2 hours duration. mGluR2/3 agonists of use in the present invention are disclosed in e.g., WO 99/003822, WO 00/200595, WO 00/029371, WO 00/075101, WO 00/075102, WO 00/132644, WO 00/156990, WO 00/192212, WO 00/192213. Specific examples of mGluR2/3 agonists include LY-181837, LY-354740, LY-379268, LY-389795, LY-404039, LY-446433, LY-459477, LY-544344, LY-2140023, talaglumetad, MGS0028 and MGS0039.

By the term “mGluR2 agonist” is meant any exogenously administered compound or agent that augments the activity of the mGluR2 receptor, in particular, the human mGluR2 receptor. mGluR2 agonists suitable for use in the present invention may be identified without undue experimentation by methodology well known in the art (see for example Monn et al., (1999) J. Med Chem 42(6), 1027-1040).

In an embodiment of the present invention the mGluR2 agonist possesses a selectivity for the mGluR2 receptor relative to ionotropic glutamate receptors of at least 5 fold as measured by the ratio of EC50 for the mGluR2 receptor to the EC50 for ionotropic glutamate receptors as evaluated by the above assays. In an embodiment of the present invention the mGluR2 agonist possesses a selectivity for the mGluR2 receptor relative to ionotropic glutamate receptors of at least 10 fold as measured by the ratio of EC50 for the mGluR2 receptor to the EC50 for ionotropic glutamate receptors as evaluated by the above assays. In an embodiment of the present invention the mGluR2 agonist possesses a selectivity for the mGluR2 receptor relative to ionotropic glutamate receptors of at least 50 fold as measured by the ratio of EC50 for the mGluR2 receptor to the EC50 for ionotropic glutamate receptors as evaluated by the above assays. In an embodiment of the present invention the mGluR2 agonist possesses a selectivity for the mGluR2 receptor relative to ionotropic glutamate receptors of at least 100 fold as measured by the ratio of EC50 for the mGluR2 receptor to the EC50 for ionotropic glutamate receptors as evaluated by the above assays.

In an embodiment of the present invention the mGluR2 agonist possesses a selectivity for the mGluR2 receptor relative to each of the other mGluR receptors of at least 5 fold as measured by the ratio of EC50 for the mGluR2 receptor to the EC50 for each of the other mGluR receptors as evaluated by the above assays. In an embodiment of the present invention the mGluR2 agonist possesses a selectivity for the mGluR2 receptor relative to each of the other mGluR receptors of at least 10 fold as measured by the ratio of EC50 for the mGluR2 receptor to the EC50 for each of the other mGluR receptors as evaluated by the above assays. In an embodiment of the present invention the mGluR2 agonist possesses a selectivity for the mGluR2 receptor relative to each of the other mGluR receptors of at least 50 fold as measured by the ratio of EC50 for the mGluR2 receptor to the EC50 for each of the other mGluR receptors as evaluated by the above assays. In an embodiment of the present invention the mGluR2 agonist possesses a selectivity for the mGluR2 receptor relative to each of the other mGluR receptors of at least 100 fold as measured by the ratio of EC50 for the mGluR2 receptor to the EC50 for each of the other mGluR receptors as evaluated by the above assays.

In an aspect of the present invention the mGluR2 agonist possesses an EC50 for binding to the mGluR2 receptor of 1 uM or less as evaluated by the above assays. In an aspect of the present invention the mGluR2 agonist possesses an EC50 for binding to the mGluR2 receptor of 500 nM or less as evaluated by the above assays. In an aspect of the present invention the mGluR2 agonist possesses an EC50 for binding to the mGluR2 receptor of 100 nM or less as evaluated by the above assays. In an aspect of the present invention the mGluR2 agonist possesses an EC50 for binding to the mGluR2 receptor of 50 nM or less as evaluated by the above assays. In an aspect of the present invention the mGluR2 agonist possesses an EC50 for binding to the mGluR2 receptor of 10 nM or less as evaluated by the above assays.

In an embodiment of the present invention the mGluR2 agonist is an orally active mGluR2 agonist. In an embodiment of the present invention the mGluR2 agonist is orally administered. In another embodiment of the present invention the mGluR2 agonist is a non-peptidal mGluR2 agonist. In an embodiment of the present invention the mGluR2 agonist is a CNS-penetrant mGluR2 agonist and is able to enter the brain and/or central nervous system with sufficient concentration to have a therapeutic effect. In a further embodiment of the present invention the CNS-penetrant mGluR2 agonist is a compound that exhibits sufficient concentration in the brain and/or central nervous system to have therapeutic efficacy upon oral administration.

In an embodiment of the present invention the mGluR2 agonist has an onset of action of 45-60 minutes. In an embodiment of the present invention the mGluR2 agonist has a pharmacological half life (T½ life) of short duration. In another embodiment of the present invention the mGluR2 agonist has a pharmacological half life (T½ life) of intermediate duration. In another embodiment of the present invention the mGluR2 agonist has a pharmacological half life (T½ life) of long duration. In another embodiment of the present invention the mGluR2 agonist has a pharmacological half life (T½ life) of at least about 2 hours duration.

By the term “mGluR2 potentiator” is meant any exogenously administered compound or agent that directly or indirectly augments the response produced by the endogenous ligand (such as glutamate) at the mGluR2 receptor, in particular, the human mGluR2 receptor. mGluR2 potentiators suitable for use in the present invention may be identified without undue experimentation by methodology well known in the art (e.g. US 2005/0069498). For example, the compound may be tested in a [³⁵S]-GTPyS assay. The stimulation of [³⁵S]-GTPyS binding is a common functional assay to monitor G(alpha)i-coupled receptor in native and recombinant receptor membrane preparation. Membrane from cells stably expressing hmGlu2 CHO-K1 (50 ug) are incubated in a 96 well plate for 1 hour in the presence of GTPyS³⁵ (0.05 nM), GDP (5 uM) and compounds. The reaction is stopped by rapid filtration over Unifilter GF/B plate (Packard, Bioscience, Meriden Conn.) using a 96-well cell harvester (Brandel Gaithersburg, Md.). The filter plates are counted using Topcount counter (Packard, Bioscience, Meriden Conn., USA). When compounds are evaluated as potentiator they are tested in the presence of glutamate (1 uM). The activation (agonist) or the potentiation of glutamate (potentiator) curves are fitted with a four parameters logistic equation giving EC₅₀ and Hill coefficient using the iterative non linear curve fitting software GraphPad (San Diego Calif., USA).

In an embodiment of the present invention the mGluR2 potentiator possesses a selectivity for the mGluR2 receptor relative to each of the other mGluR receptors of at least 5 fold as measured by the ratio of EC50 for the mGluR2 receptor to the EC50 for each of the other mGluR receptors as evaluated by the above assay. In an embodiment of the present invention the mGluR2 potentiator possesses a selectivity for the mGluR2 receptor relative to each of the other mGluR receptors of at least 10 fold as measured by the ratio of EC50 for the mGluR2 receptor to the EC50 for each of the other mGluR receptors as evaluated by the above assay. In an embodiment of the present invention the mGluR2 potentiator possesses a selectivity for the mGluR2 receptor relative to each of the other mGluR receptors of at least 50 fold as measured by the ratio of EC50 for the mGluR2 receptor to the EC50 for each of the other mGluR receptors as evaluated by the above assay. In an embodiment of the present invention the mGluR2 potentiator possesses a selectivity for the mGluR2 receptor relative to each of the other mGluR receptors of at least 100 fold as measured by the ratio of EC50 for the mGluR2 receptor to the EC50 for each of the other mGluR receptors as evaluated by the above assay.

In an aspect of the present invention the mGluR2 potentiator possesses an EC50 for binding to the mGluR2 receptor of 1 uM or less as evaluated by the above assay. In an aspect of the present invention the mGluR2 potentiator possesses an EC50 for binding to the mGluR2 receptor of 500 nM or less as evaluated by the above assay. In an aspect of the present invention the mGluR2 potentiator possesses an EC50 for binding to the mGluR2 receptor of 100 nM or less as evaluated by the above assay. In an aspect of the present invention the mGluR2 potentiator possesses an EC50 for binding to the mGluR2 receptor of 50 nM or less as evaluated by the above assay. In an aspect of the present invention the mGluR2 potentiator possesses an EC50 for binding to the mGluR2 receptor of 10 nM or less as evaluated by the above assay.

In an embodiment of the present invention the mGluR2 potentiator is an orally active mGluR2 potentiator. In an embodiment of the present invention the mGluR2 potentiator is orally administered. In another embodiment of the present invention the mGluR2 potentiator is a non-peptidal mGluR2 potentiator. In an embodiment of the present invention the mGluR2 potentiator is a CNS-penetrant mGluR2 potentiator and is able to enter the brain and/or central nervous system with sufficient concentration to have a therapeutic effect. In a further embodiment of the present invention the CNS-penetrant mGluR2 potentiator is a compound that exhibits sufficient concentration in the brain and/or central nervous system to have therapeutic efficacy upon oral administration.

In an embodiment of the present invention the mGluR2 potentiator has an onset of action of 45-60 minutes. In an embodiment of the present invention the mGluR2 potentiator has a pharmacological half life (T½ life) of short duration. In another embodiment of the present invention the mGluR2 potentiator has a pharmacological half life (T½ life) of intermediate duration. In another embodiment of the present invention the mGluR2 potentiator has a pharmacological half life (T½ life) of long duration. In another embodiment of the present invention the mGluR2 potentiator has a pharmacological half life (T½ life) of at least about 2 hours duration. mGluR2 potentiators of use in the present invention are disclosed in e.g., WO 2001/056990, WO 2004/018386, WO 2004/092135, WO 2006/014918, WO 2006/015158, WO 2006/030031, WO 2006/047237, WO 2006/049968, WO 2006/057870, WO 2006/091496, WO 2007/021309, WO 2007/078523, EP1809608, US 2007/0066573.

The subject treated in the present methods is generally a mammal, in particular, a human being, male or female, in whom therapy is desired. The term “therapeutically effective amount” means the amount of the compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with such disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy to retard the progression or reduce the risk of the noted conditions, particularly in a patient who is predisposed to such disease or disorder. In an embodiment of the present invention, the patient is a human. Within this embodiment, the patient is a human who exhibits poor response to therapy with atypical antipsychotics.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particular embodiments include the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particular embodiments citric, hydrobromic, hydrochloric, maleic, phosphoric, sulfuric, fumaric, and tartaric acids. It will be understood that, as used herein, references to compounds within the combination of the present invention are meant to also include the pharmaceutically acceptable salts.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof. The terms “administration of” and or “administering a” compound should be understood to mean providing a compound of the invention or a prodrug of a compound of the invention to the individual in need of treatment.

As used herein, the expression “in combination with” requires that therapeutically effective amounts of both the 5-HT2A antagonist or a pharmaceutically acceptable salt thereof, to the mGluR2 agonist, mGluR2/3 agonist or mGluR2 potentiator or a pharmaceutically acceptable salt thereof, are administered to the subject, but places no restriction on the manner in which this is achieved. Thus, the two species may be combined in a single dosage form for simultaneous administration to the subject, or may be provided in separate dosage forms for contemporaneous or sequential administration to the subject. When the species of the combination of the present invention are used simultaneously, a pharmaceutical composition in unit dosage form containing such species of the combination of the present invention may be desireable. Sequential administration may be close in time or remote in time, e.g. one species administered in the morning and the other in the evening. Therapy may also includes regimens in which the species of the combination of the present invention are administered on different overlapping schedules. The separate species may be administered at the same frequency or at different frequencies, e.g. one species once a day and the other two or more times a day. The separate species may be administered by the same route or by different routes, e.g. one species orally and the other parenterally, although oral administration of both species is preferred, where possible. It is also contemplated that in the combination of the present invention, the species of the combination may be used in lower doses than when each is used singly.

The weight ratio of the 5-HT2A antagonist or a pharmaceutically acceptable salt thereof, to the mGluR2 agonist, mGluR2/3 agonist or mGluR2 potentiator or a pharmaceutically acceptable salt thereof, within the combination of the present invention may be varied and will depend upon the effective dose of each ingredient. Generally, an effective dose of each will be used. Thus, for example, when a 5-HT2A antagonist or a pharmaceutically acceptable salt thereof, is combined with an mGluR2 agonist, mGluR2/3 agonist or mGluR2 potentiator or a pharmaceutically acceptable salt thereof, the weight ratio of the 5-HT2A antagonist or a pharmaceutically acceptable salt thereof, to the mGluR2 agonist, mGluR2/3 agonist or mGluR2 potentiator or a pharmaceutically acceptable salt thereof, will generally range from about 1000:1 to about 1:1000, such as about 200:1 to about 1:200. Combinations of a compound of the present invention and other active ingredients will generally also be within the aforementioned range, but in each case, an effective dose of each active ingredient should be used.

With respect to the use of a 5-HT2A antagonist, an mGluR2 agonist, an mGluR2/3 agonist or an mGluR2 potentiator alone, the present combination of a 5-HT2A antagonist, and an mGluR2 agonist, an mGluR2/3 agonist or an mGluR2 potentiator, enables treatment of psychosis and other neurological and psychiatric disorders while decreasing the side effects (such as extrapyramidal side effects) seen with other agents, including typical neuroleptics such as haloperidol. It is believed that the combination of medicaments, acting through separate mechanisms, provides an unexpected effect, enabling use of sub-therapeutic doses of the separate components (or other agents) in favorable cases. In accordance with the present invention, whereas certain doses of a 5-HT2A antagonist and certain doses an mGluR2 agonist, an mGluR2/3 agonist or an mGluR2 potentiator are ineffecive alone, when such doses of a 5-HT2A antagonist and such doses an mGluR2 agonist, an mGluR2/3 agonist or an mGluR2 potentiator are surprisingly effective when administered in combination with each other. The combination of the present invention is expected to provide greater efficacy on positive and negative symptoms of schizophrenia. The combination of the present invention is also expected to be relatively well tolerated and have a lower propensity for weight gain and treatment related metabolic disorders relative to currently-marketed atypical antipsychotics.

The combination of the present invention has utility in treating a variety of neurological and psychiatric disorders, including one or more of the following conditions or diseases: schizophrenia or psychosis including schizophrenia (paranoid, disorganized, catatonic or undifferentiated), schizophreniform disorder, schizoaffective disorder, severe schizoaffective disorder with psychotic features, delusional disorder, brief psychotic disorder, shared psychotic disorder, psychotic disorder due to a general medical condition and substance-induced or drug-induced (phencyclidine, ketamine and other dissociative anaesthetics, amphetamine and other psychostimulants and cocaine), psychosispsychotic disorder, psychosis associated with affective disorders, brief reactive psychosis, schizoaffective psychosis, “schizophrenia-spectrum” disorders such as schizoid or schizotypal personality disorders, schizotypical personality disorders with sedative, hypnotic or anxiolytic manifestiations, paranoid personality disorders, or illness associated with psychosis (such as major depression, manic depressive (bipolar) disorder, Alzheimer's disease and post-traumatic stress syndrome), including both the positive and the negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence, relapse prevention or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders (including bipolar I disorder with single manic episode, bipolar I disorder with psychotic features, bipolar I disorders manifesting a mixed most recent episode, severe bipolar I disorders with psychotic features), mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; NMDA receptor-related disorders such as autism, depression, benign forgeffulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias [including tremor (such as rest tremor, postural tremor and intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics),and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

Of the disorders above, the treatment of schizophrenia, bipolar disorder, depression including unipolar depression, seasonal depression and post-partum depression, premenstrual syndrome (PMS) and premenstrual dysphoric disorder (PDD), learning disorders, pervasive developmental disorder including autistic disorder, attention disorders including Attention-Deficit/Hyperactivity Disorder, autism,tic disorders including Tourette's disorder, anxiety disorders including phobia and post traumatic stress disorder, cognitive disorders associated with dementia, AIDS dementia, Alzheimer's, Parkinson's, Huntington's disease, spasticity, myoclonus, muscle spasm, tinnitus and hearing impairment and loss are of particular importance.

In another specific embodiment, the present invention provides a method for treating schizophrenia or psychosis comprising: administering to a patient in need thereof an effective amount of the combination of the present invention. Particular schizophrenia or psychosis pathologies are paranoid, disorganized, catatonic or undifferentiated schizophrenia and substance-induced psychotic disorder. At present, the text revision of the fourth edition of the Diagnostic and Statistical Manual of Mental Disorders (DSM-IV-TR) (2000, American

Psychiatric Association, Washington D.C.) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources. In general, schizophrenia refers to a psychopathic disorder of unknown origin, which usually appears for the first time in early adulthood and is marked by a number of characteristics, psychotic symptoms, progression, phasic development and deterioration in social behaviour and professional capability in the region below the highest level ever attained. Characteristic psychotic symptoms are disorders of thought content (multiple, fragmentary, incoherent, implausible or simply delusional contents or ideas of persecution) and of mentality (loss of association, flight of imagination, incoherence up to incomprehensibility), as well as disorders of perceptibility (hallucinations), of emotions (superficial or inadequate emotions), of self-perception, of intentions and impulses, of interhuman relationships, and finally psychomotoric disorders (such as catatonia). Other symptoms are also associated with this disorder. (See, American Statistical and Diagnostic Handbook). Use of the combination of the present invention enables treatment of schizophrenia without the side effects (in particular extrapyramidal side effects) seen with typical neuroleptics such as haloperidol, and it is believed that the combination of medicaments, acting through separate mechanisms, provides an unexpected effect, enabling use of sub-therapeutic doses of the separate components (or other agents) in favorable cases.

In a specific embodiment, the present invention provides a method for treating cognitive disorders, comprising: administering to a patient in need thereof an effective amount of the combination of the present invention. Particular cognitive disorders are dementia, delirium, amnestic disorders and age-related cognitive decline. At present, the text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In another specific embodiment, the present invention provides a method for treating anxiety disorders, comprising: administering to a patient in need thereof an effective amount of the combination of the present invention. Particular anxiety disorders are generalized anxiety disorder, obsessive-compulsive disorder and panic attack. At present, the text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In another specific embodiment, the present invention provides a method for treating substance-related disorders and addictive behaviors, comprising: administering to a patient in need thereof an effective amount of the combination of the present invention. Particular substance-related disorders and addictive behaviors are persisting dementia, persisting amnestic disorder, psychotic disorder or anxiety disorder induced by substance abuse; and tolerance of, dependence on or withdrawal from substances of abuse. At present, the text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

In another specific embodiment, the present invention provides a method for treating pain, comprising: administering to a patient in need thereof an effective amount of the combination of the present invention. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

In another specific embodiment, the present invention provides a method for treating obesity or eating disorders associated with excessive food intake and complications associated therewith, comprising: administering to a patient in need thereof an effective amount of the combination of the present invention. At present, obesity is included in the tenth edition of the International Classification of Diseases and Related Health Problems (ICD-10) (1992 World Health Organization) as a general medical condition. The text revision of the fourth edition of the Diagnostic and Statistical Manual of Mental Disorders (DSM-IV-TR) (2000, American Psychiatric Association, Washington D.C.) provides a diagnostic tool that includes obesity in the presence of psychological factors affecting medical condition. As used herein, the term “obesity or eating disorders associated with excessive food intake” includes treatment of those medical conditions and disorders described in ICD-10 and DSM-IV-TR. The skilled artisan will recognize that there are alternative nomenclatures, nosologies and classification systems for general medical conditions, and that these systems evolve with medical and scientific progress. Thus the term “obesity or eating disorders associated with excessive food intake” is intended to include like conditions and disorders that are described in other diagnostic sources.

The subject combination is further useful in a method for the prevention, treatment, control, amelioration, or reducation of risk of the diseases, disorders and conditions noted herein. The subject combination is further useful in a method for the prevention, treatment, control, amelioration, or reduction of risk of the aforementioned diseases, disorders and conditions in combination with other agents. The combination of the present invention may be used in conjunction with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefor, contemporaneously or sequentially with the combination of the present invention. When the combination of the present invention is used contemporaneously with one or more other drugs, a pharmaceutical composition in unit dosage form containing such other drugs and the combination of the present invention may be desireable. However, such conjunctive therapy may also includes therapies in which the combination of the present invention and one or more other drugs are administered on different overlapping schedules. It is also contemplated that when used in further conjunction with one or more other active ingredients, the combination of the present invention and the other active ingredients may be used in lower doses than when each is used singly. Accordingly, the pharmaceutical compositions of the present invention include those that also contain one or more other active ingredients, in addition to the combination of the present invention. The weight ratio of the compounds within theof the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, such as about 200:1 to about 1:200. Combinations of a compound of the present invention and other active ingredients will generally also be within the aforementioned range, but in each case, an effective dose of each active ingredient should be used.

In such further combinations the combination of the present invention and other active agents may be administered separately or in conjunction. In addition, the administration of one element may be prior to, concurrent to, or subsequent to the administration of other agent(s).

Accordingly, the subject combination may be used alone or in further combination with other agents which are known to be beneficial in the subject indications or other drugs that affect receptors or enzymes that either increase the efficacy, safety, convenience, or reduce unwanted side effects or toxicity of the compounds of the present invention. The subject combination and the other agent may be co-administered, either in concomitant therapy or in a fixed combination.

In one embodiment, the subject combination may be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies.

In another embodiment, the subject combination may be employed in further combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject combination may be administered in conjunction with the use of physical methods such as with light therapy or electrical stimulation.

In another embodiment, the subject combination may be employed in further combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In another embodiment, the subject combination may be employed in further combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject combination may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject combination may be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In another embodiment, the subject combination may be employed in further combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

The combination of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the combination of the invention is effective for use in humans.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. Accordingly, the pharmaceutical compositions of the present invention encompass any composition made by admixing a compound of the present invention and a pharmaceutically acceptable carrier. Depending on whether they are to be administered together or separately, the 5-HT2A antagonist or a pharmaceutically acceptable salt thereof, and an mGluR2 agonist, an mGluR2/3 agonist or an mGluR2 potentiator or a pharmaceutically acceptable salt thereof, are typically supplied as single or multiple pharmaceutical compositions comprising the active species and a pharmaceutically acceptable carrier.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Aqueous suspensions, oily suspensions, dispersible powders or granules, oil-in-water emulsions, and sterile injectable aqueous or oleagenous suspension may be prepared by standard methods known in the art.

The subject combinations are further useful in a method for the prevention, treatment, control, amelioration, or reducation of risk of the diseases, disorders and conditions noted herein. The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 10 mg/kg. of body weight daily are administered to the patient, e.g., humans and elderly humans. The dosage range will generally be about 0.5 mg to 1.0 g. per patient per day which may be administered in single or multiple doses. In one embodiment, the dosage range will be about 0.5 mg to 500 mg per patient per day; in another embodiment about 0.5 mg to 200 mg per patient per day; and in yet another embodiment about 5 mg to 50 mg per patient per day. Pharmaceutical compositions of the present invention may be provided in a solid dosage formulation such as comprising about 0.5 mg to 500 mg active ingredient, or comprising about 1 mg to 250 mg active ingredient. The pharmaceutical composition may be provided in a solid dosage formulation comprising about 1 mg, 5 mg, 10 mg, 25 mg, 50 mg, 100 mg, 200 mg or 250 mg active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, such as once or twice per day.

The following examples are provided so that the invention might be more fully understood. These examples are illustrative only and should not be construed as limiting the invention in any way.

### Example 1

### Effect of an mGluR 2/3 agonist and a 5-HT2a Antagonist on Ketamine-Induced Psychomotor Activity

Compounds with antipsychotic efficacy are known to block Ketamine-induced psychomotor activity in rats (Bardi et al., Behavioural Pharmacology (2007), 18(2), 103-118). Male Wistar rats were received on site and housed for 7 days with food and water ad libitum. On the day of testing, animals were given vehicle (water for mGluR2/3 agonist, 1% Tween for 5-HT2A antagonist), the 5-HT2A antagonist volinanserin (0.2 mpk), the mGluR2/3 agonist (−)-2-oxa-4-aminobicyclo[3.1.0]hexane-4,6-dicarboxylate (1 mpk), or both the 5-HT2A antagonist (0.2 mpk) and the mGluR2/3 agonist (1 mpk) and placed in locomotor activity monitors. Thirty minutes later animals received a subcutaneous injection of saline or 37.5 mg/kg Ketamine and placed back into the'locomotor activity monitors for 90 minutes. Total distance traveled was used to assess locomotor behavior. The distance traveled is summed over the ninety minutes after ketamine or saline treatment. ANOVA analysis revealed a main effect of group.

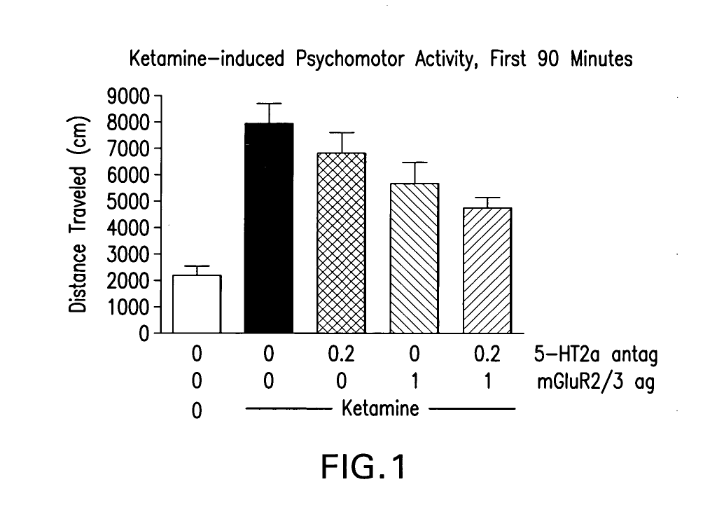

As shown in FIG. 1, ketamine increased psychomotor activity in all groups, however the mGluR2/3 agonist alone or the combination of the 5-HT2A antagonist+the mGluR2/3 agonist decreased the response to ketamine, relative to the 0-0-Ketamine group. Surprisingly, the combination of 5-HT2A antagonist+the mGluR2/3 agonist decreased the response to ketamine, relative to the group receiving the 5-HT2A antagonist alone.

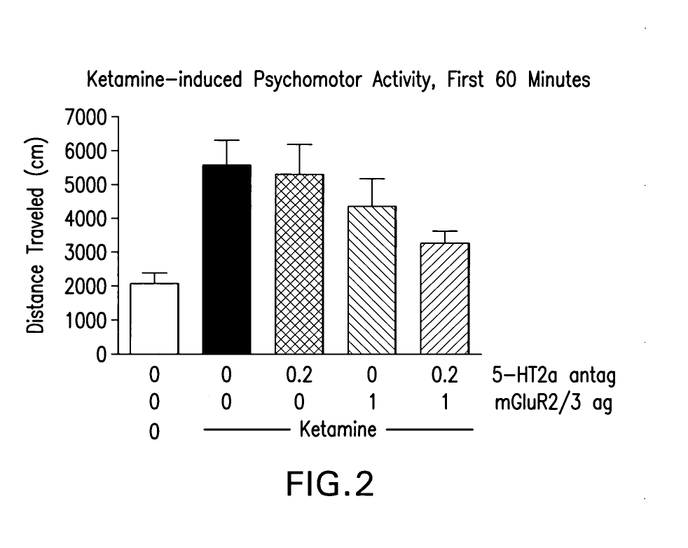

Similarly, locomotor response was determined over the first 60 minutes after receiving ketamine or saline as a function of treatment with vehicle, the 5-HT2a antagonist (0.2 mpk), the mGluR2/3 agonist (1 mpk), or the 5-HT2a antagonist (0.2 mpk)+the mGluR2/3 agonist (1 mpk). ANOVA analysis revealed a main effect of group. As shown in FIG. 2, ketamine increased psychomotor activity in the ketamine group, the group receiving the 5-HT2a antagonist alone and the group receiving the mGluR2/3 agonist alone. Surprisingly, in the group receiving both the 5-HT2a antagonist+the mGluR2/3 agonist, ketamine did not increase psychomotor activity. In addition, the group receiving both the 5-HT2a antagonist+the mGluR2/3 agonist prior to ketamine was the only ketamine-treated group that significantly differed from the group receiving ketamine alone.

### Example 2

### Effect of an mGluR 2/3 Agonist and a 5-HT2a Antagonist on Amphetamine-Induced Psychomotor Activity

Compounds with antipsychotic efficacy are known to block amphetamine-induced psychomotor activity in rats (Bardi et al., Behavioural Pharmacology (2007), 18(2), 103-118). Male Wistar rats were received on site and housed for 7 days with food and water ad libitum. On the day of testing, animals were given vehicle (water for mGluR2/3 agonist, 1% Tween for 5-HT2A antagonist), the 5-HT2A antagonist volinanserin (0.2 mpk), the mGluR2/3 agonist (−)-2-oxa-4-aminobicyclo[3.1.0]hexane-4,6-dicarboxylate (1 mpk), or both the 5-HT2A antagonist (0.2 mpk) and the mGluR2/3 agonist (1 mpk) and placed in locomotor activity monitors. Thirty minutes later animals received a subcutaneous injection of saline or 1.5 mg/kg amphetamine and placed back into the locomotor activity monitors for 90 minutes. Total distance traveled was used to assess locomotor behavior. The distance traveled is summed over the ninety minutes after amphetamine or saline treatment. ANOVA analysis revealed a main effect of group.

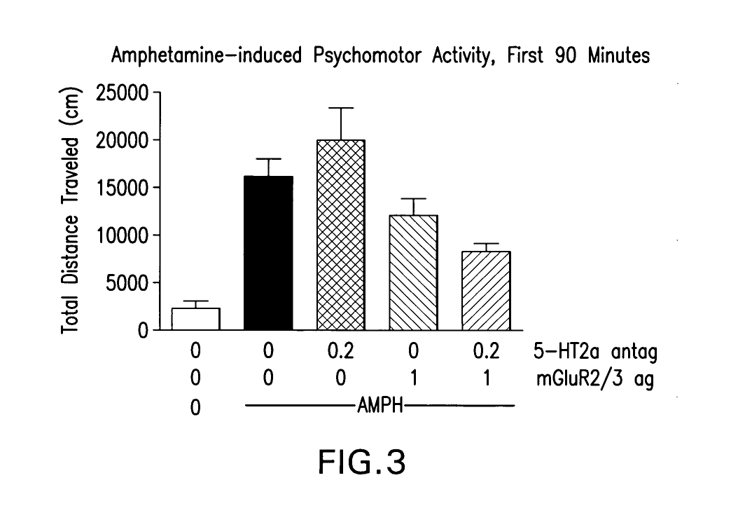

As shown in FIG. 3, ketamine increased psychomotor activity in the amphetamine group, the group receiving the 5-HT2a antagonist alone and the group receiving the mGluR2/3 agonist alone. Surprisingly, in the group receiving both the 5-HT2a antagonist+the mGluR2/3 agonist, amphetamine did not increase psychomotor activity. In fact, the group receiving both the 5-HT2a antagonist+the mGluR2/3 agonist did not differ significantly from the group receiving just vehicle. When given alone, the 5-HT2a antagonist or the mGluR2/3 agonist did not influence amphetamine-induced psychomotor activity at the doses tested. Surprisingly, when the 5-HT2a antagonist and the mGluR2/3 agonist were administered in combination, they significantly reduced the psychomotor activity produced by amphetamine.

### Example 3

### Effect of an mGluR 2/3 Agonist and a 5-HT2a Antagonist on MK-801-Induced Psychomotor Activity

Compounds with antipsychotic efficacy are known to block MK-801-induced psychomotor activity in rats (Bardi et al., Behavioural Pharmacology (2007), 18(2), 103-118). Male Wistar rats were received on site and housed for 7 days with food and water ad libitum. On the day of testing, animals were given vehicle (water for mGluR2/3 agonist, 1% Tween for 5-HT2A antagonist), the 5-HT2A antagonist volinanserin (0.2 mpk), the mGluR2/3 agonist (−)-2-oxa-4-aminobicyclo[3.1.0]hexane-4,6-dicarboxylate (1 mpk), or both the 5-HT2A antagonist (0.2 mpk) and the mGluR2/3 agonist (1 mpk) and placed in locomotor activity monitors. Thirty minutes later animals received a subcutaneous injection of saline or 0.2 mg/kg MK-801 and were placed back into the locomotor activity monitors for 90 minutes. Total distance traveled was used to assess locomotor behavior. The distance traveled is summed over the ninety minutes after MK-801 or saline treatment. ANOVA analysis revealed a main effect of group.

In particular, MK-801 increased psychomotor activity in all groups (control≃200 cm; MK-801 alone≃2000 cm; MK-801+mGluR2/3 agonist≃1900 cm; MK-801+5-HT2A antagonist≃2500 cm; MK-801+mGluR2/3 agonist+5-HT2A antagonist≃1100 cm). Neither the mGluR2/3 agonist nor the 5-HT2A antagonist given alone reduced the response to MK-801. However, when given together, the mGluR2/3 agonist and the 5-HT2A antagonist significantly reduced the response to MK-801 (MK-801 alone≃2000 cm; MK-801+mGluR2/3 agonist+5-HT2A antagonist≃1100 cm). Indeed, the combination of the mGluR2/3 agonist and the 5-HT2A antagonist reduced the MK-801 response more than would have been expected by mere additivity (i.e. if additive: MK-801 alone+(MK-801+mGluR2/3 agonist)+(MK-801 +5-HT2A antagonist≃2400 cm. Obeserved: MK-801+mGluR2/3 agonist+5-HT2A antagonist≃1100 cm).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A combination which comprises a 5-HT2A antagonist or a pharmaceutically acceptable salt thereof, and an mGluR2/3 agonist, an mGluR2 agonist or an mGluR2 potentiator or a pharmaceutically acceptable salt thereof.

2. The combination of claim 1 which comprises an mGluR2/3 agonist or a pharmaceutically acceptable salt thereof.

3. The combination of claim 1 which comprises an mGluR2 agonist or a pharmaceutically acceptable salt thereof.

4. The combination of claim 1 which comprises an mGluR2 potentiator or a pharmaceutically acceptable salt thereof.

5. The combination of claim 1 wherein the 5-HT2A antagonist is selective for the human 5-HT2A receptor over one or more of the human 5-HT2C receptor, dopamine receptors and IKr.

6. The combination of claim 1 wherein the 5-HT2A antagonist possesses a selectivity for the 5-HT2A receptor relative to each of the other 5-HT2 receptors of at least 5 fold as measured by the ratio of IC50 for the 5-HT2A receptor to the IC50 for each of the other 5-HT2 receptors.

7. The combination of claim 1 wherein the 5-HT2A antagonist possesses a selectivity for the 5-HT2A receptor relative to the dopamine D2 receptor of at least 5 fold as measured by the ratio of IC50 for the 5-HT2A receptor to the IC50 for the dopamine D2 receptor.

8. The combination of claim 1 wherein the 5-HT2A antagonist possesses an IC50 for blocking the 5-HT2A antagonist receptor of 500 nM or less.

9. The combination of claim 2 wherein the mGluR2/3 agonist possesses a selectivity for the mGluR2 receptor and the mGluR3 receptor relative to ionotropic glutamate receptors of at least 5 fold as measured by the ratio of EC50 for the mGluR2 receptor and the mGluR3 receptor to the EC50 for ionotropic glutamate receptors.

10. The combination of claim 2 wherein the mGluR2/3 agonist possesses an EC50 for binding to the mGluR2 receptor and the mGluR3 receptor of 500 nM or less.

11. The combination of claim 3 wherein the mGluR2 agonist possesses a selectivity for the mGluR2 receptor relative to ionotropic glutamate receptors of at least 5 fold as measured by the ratio of EC50 for the mGluR2 receptor to the EC50 for ionotropic glutamate receptors.

12. The combination of claim 3 wherein the mGluR2 agonist possesses an EC50 for binding to the mGluR2 receptor and the mGluR3 receptor of 500 nM or less.

13. A pharmaceutical composition which comprises a pharmaceutically acceptable carrier, a 5-HT2A antagonist or a pharmaceutically acceptable salt thereof, and an mGluR2/3 agonist, an mGluR2 agonist or an mGluR2 potentiator or a pharmaceutically acceptable salt thereof.

14. (canceled)

15. A method for treating psychosis in a mammalian patient in need thereof which comprises administering to the patient a therapeutically effective amount of the combination of claim 1.

16. A method for treating schizophrenia in a mammalian patient in need thereof which comprises administering to the patient a therapeutically effective amount of the combination of claim 1.

17. A method for treating bipolar disorder in a mammalian patient in need thereof which comprises administering to the patient a therapeutically effective amount of the combination of claim 1.

18. A method for treating psychosis in Alzheimer's disease in a mammalian patient in need thereof which comprises administering to the patient a therapeutically effective amount of the combination of claim 1.

19. A method for enhancing cognition in a mammalian patient in need thereof which comprises administering to the patient a therapeutically effective amount of the combination of claim 1.
